(19) United States
(12) Patent Application Publication
(10) Pub. No.: US 2005/0242103 A1
(43) Pub. Date: Nov. 3, 2005

(54) Insulated color-changing drinking cup

(76) Inventors: Sherry L. Thomas, Hull, MA (US); John A. Hession, Braintree, MA (US)

Correspondence Address: FISH & RICHARDSON PC, P.O. BOX 1022, MINNEAPOLIS, MN, 55440-1022, US

(21) Appl. No.: 10/836,019

(22) Filed: Apr. 29, 2004

(51) Int. Cl. A61K 9/48

(52) U.S. Cl. 220/717

(57) ABSTRACT

A drinking cup includes an inner wall with an outer surface and an inner surface. The inner wall includes a thermo-chromatic substance that exhibits a color variance corresponding to a temperature variance in a beverage in the cup that is in contact with the inner wall. An outer wall is disposed about the inner wall that has a transparent area adapted to permit a visual perception of at least a portion of the color variance of the thermo-chromatic substance in or on the inner wall through the outer wall. A thermal insulation layer is disposed between the inner wall and the outer wall.

### TECHNICAL FIELD

This invention relates to spill-resistant cups, and more particularly to a children's drinking cup that can change colors.

### BACKGROUND

Children, especially infants and toddlers, have specific nutritional needs. Often, these needs are for specific liquids. During this developmental time, however, it is often desirable to present nutrition to children in a way that fosters independence and self-reliance. Spill-resistant cups have been used in the past to provide children with nutrition while simultaneously preventing the child from accidental spillage of the contents of the cup. Additionally, many liquids that toddlers consume are required to be given to the children at a required temperature. Caregivers often attempt to present the beverage or contents of the cup at a particular temperature, however a mistake in preparing the contents can lead to a temperature that is either too hot or not warm enough. Alternatively, the temperature of the beverage may be cool or even cold, but over time the beverage may warm to room temperature.

Generally, thermo-chromatic substances display a color variance based on a temperature variance. The color variance of a particular thermo-chromatic substance may be changed along with the properties of the substance. In other words, various ranges of temperatures may be used to affect a color variance in a particular thermo-chromatic substance. Some thermo-chromatic substances exhibit a color variance upon the introduction of a substance colder than the temperature threshold of the particular thermo-chromatic substance. Other thermo-chromatic substances exhibit a color variance based on the introduction of a substance with a higher temperature or hotter than the temperature threshold of the thermo-chromatic substance. If the thermo-chromatic substance is used in a cup or other drinking vessel, the act of grasping the vessel may impart sufficient heat to the surface, and thus the thermo-chromatic substance affixed to or impregnated within the surface to reduce, eliminate, or exaggerate the thermo-chromatic properties.

### SUMMARY

A drinking cup includes an inner wall having an outer surface and an inner surface. The inner wall includes a thermo-chromatic substance that may exhibit a color variance corresponding to a temperature variance in a beverage in the cup that is in contact with the inner wall. An outer wall is disposed about the inner wall that has a transparent area that allows someone to visually perceive at least a portion of the inner wall through the outer wall. The visual perception includes exhibition of the color variance, and a thermal insulation layer is disposed between the inner wall and the outer wall.

The thermal insulation layer may be air or some other type of gas. Alternatively, the thermal insulation layer may be a vacuum or a space between the inner and outer walls that is substantially void of air and/or other gases.

In some configurations, the outer wall may be molded from a clear resin. The thermo-chromatic substance may be disposed in a layer on the outer surface of the inner layer. Alternatively, the thermo-chromatic substance may be a part of the material that comprises the inner wall.

Certain implementations may include a design disposed in a layer affixed to the outer surface of the inner wall. In yet another implementation, a graphic design may be formed by an absence of the thermo-chromatic substance in a portion of the inner wall. The drinking cup may also include a lid that resists spillage of the contents. The lid may include a valve and/or an air vent that is adapted to prevent a beverage disposed within the inner wall from exiting a space bounded by the inner wall and the lid except through the spout.

Certain implementations of the invention may include one or more of the following advantages. A thermo-chromatic substance may allow a visual perception of the temperature of the contents of the cup. Additionally, a graphic design may allow the cup to display a warning if the cup contains a beverage that has an improper temperature for the intended recipient of the contents. Additionally, the insulation layer may allow a user to grasp the outer layer and manipulate the cup without directly contacting, and thereby directly transferring energy in the form of body heat, to the inner layer and/or the beverage in contact with the inner layer.

The details of one or more embodiments of the invention are set forth in the accompanying drawings and the description below. Other features, objects, and advantages of the invention will be apparent from the description and drawings, and from the claims.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a side view of an implementation of a drinking cup with a cut-away portion and a design graphic.

FIG. 2A is a perspective view of a drinking cup with no color variance exhibited.

FIG. 2B is the drinking cup of FIG. 2A with a color variance exhibited that displays a message.

Like reference symbols in the various drawings indicate like elements.

### DETAILED DESCRIPTION

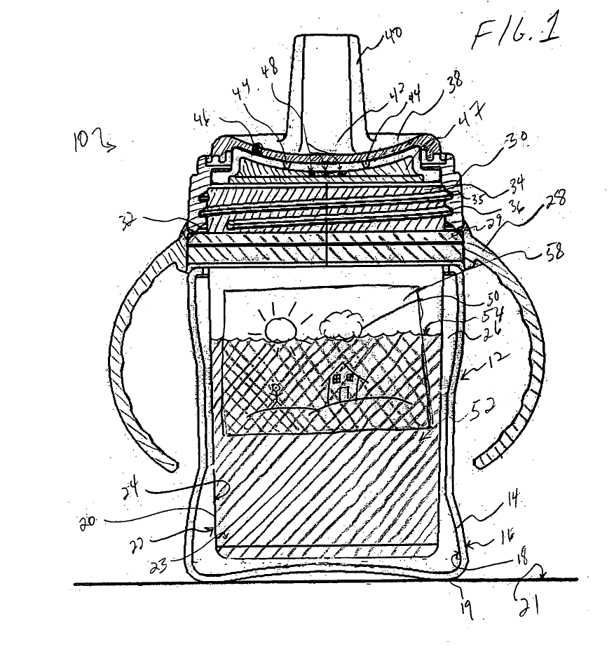

Referring to FIG. 1, a drinking cup 10 includes an outer cup 12 and a lid assembly 30. The outer cup 12 includes an outer wall 14 and a bottom surface 19. The outer wall has an exterior surface 16 and an interior surface 18, and in certain implementations, is formed to facilitate grasping of the outer wall by a child of about six months of age or older. An inner wall 20 is disposed within the interior surface 18 of the outer wall 14, and includes an outer surface 22 and an inner surface 24. The inner surface 24 may define a suitable volume. In some implementations, the volume may be between about 5 ounces and about 10 ounces. In other implementations, the inner surface 24 of the inner wall 20 may define a volume greater than about 10 ounces or less than about 5 ounces.

The outer wall 14 and/or the inner wall 20 may be manufactured from a resin, such as a thermoplastic resin, nylon, or other polymer. Additionally, the outer wall 14 is at least partially transparent, thus permitting a visual perception of at least a portion of the inner wall 20. Alternatively, the outer wall 14 may be entirely transparent, entirely translucent, or at least partially translucent. The outer wall 14 may also have a transparent, translucent, partially transparent, or partially translucent portion that permits a visual perception of at least a portion of the inner wall 20. The outer wall 14 may be a molded transparent resin that may be of unitary construction or assembled components. Additionally, the outer wall 14 may form a unitary outer cup 12 with a bottom surface 19 suitable for resting on a top surface of a table 21, or other suitable surface.

A thermal insulation layer 26 is present between the outer wall 14 and the inner wall 20. The thermal insulation layer 26 may be air, a mixture of inert gases, or a complete or partial vacuum. The thermal insulation layer 26 may limit or greatly reduce the amount of energy transferred from the outer wall 14 to the inner wall 20.

In certain implementations, a handle assembly 28 may also be coupled to an upper end 29 of the drinking cup 10. The handle assembly 28 may be molded or assembled, and may also include a male portion 34 adapted to couple a lid assembly 30 to the drinking cup 10. The handle assembly may be formed to an appropriate size that facilitates manipulation of the drinking cup 10 by a child of about six months of age or older. In the implementation shown, the male portion 34 of the handle assembly 28 includes a male threaded portion 36 adapted to rotatably couple the lid assembly 30 via a female threaded portion 36 formed in the lid assembly 30. A seal 32, such as an O-ring or other suitable seal, may be disposed between the handle assembly 28 and the lid assembly 30 to prevent leakage.

The lid assembly 30 may alternatively be coupled to the drinking cup 10 by a snap-on coupling or other suitable coupling (not explicitly shown). In certain implementations, the lid assembly 30 my incorporate a valve assembly similar to the valve assembly of U.S. patent application Ser. No. 10/648,974, which is incorporated herein by reference. In the implementation shown, the lid assembly 30 includes a flexible outer member 38 that includes a spout 40 coupled to the outer member 38 or formed as a part of the outer member 38. A channel 42 may be formed within the spout 40 that is more rigid than the spout 40 and or the outer member 38 to prevent folding of the spout 40 that may prevent or limit flow through the channel 42.

An end surface 44 may be incorporated that is substantially circular or forms a perimeter around the channel 42 of the spout 40. The end surface 44 contacts an inner member 47 when no force is acting on the spout 40. The outer member 38 may also include a vent 46 formed through the outer member 38. The inner member 47 may be more rigid than the outer member 38. One or more apertures 48 are formed through the inner member 47 that provide communication with any contents, such as a liquid or beverage 54, disposed within the inner wall 20 of the drinking cup 10. When a force acts on the spout 40, the force moves a portion of the end surface 44 away from the inner member 47 to permit the beverage 54 to flow through the apertures 48 to the channel 42 of the spout 40. The vent 46 provides air to enter the drinking cup 10 to replace the space within the inner wall 20 vacated by the beverage 54.

The inner wall 20 may be impregnated by a thermo-chromatic substance, such as a thermo-chromatic ink 52. Various methods of adding thermo-chromatic materials exist and would be suitable for application to the inner wall, such as thermo-chromatic liquid crystal ink or leucodye ink. The thermo-chromatic ink 52 may be disposed within the resin that forms the inner wall 20. Alternatively, the thermo-chromatic ink 52 may be disposed in a layer affixed to the outer surface 22 of the inner wall 20. A graphic design 50 may also be disposed on the outer surface 22 of the inner wall 20. The graphic design 50 may be fixed in a decal 58 or other device or layer disposed on the outer surface 22 of the inner wall 20. Additionally or alternatively, the decal 58 may include a thermo-chromatic ink 52 in an adhesive layer or in the decal 58 itself.

The outer wall 14 performs at least two functions. First, the transparent or translucent properties of the outer wall 14 allows a visual perception of the color variance exhibited by the thermo-chromatic ink 52 impregnated in or affixed to the inner wall 20. Additionally, the presence of the thermal insulation layer 26 between the outer wall 14 and the inner wall 20 reduce the amount of heat transfer between the inner wall 20 (and therefore the beverage 54) and the outer wall 14. The outer wall 14 also allows a visual perception of the graphic design 50 affixed to the outer surface 22 of the inner wall 20.

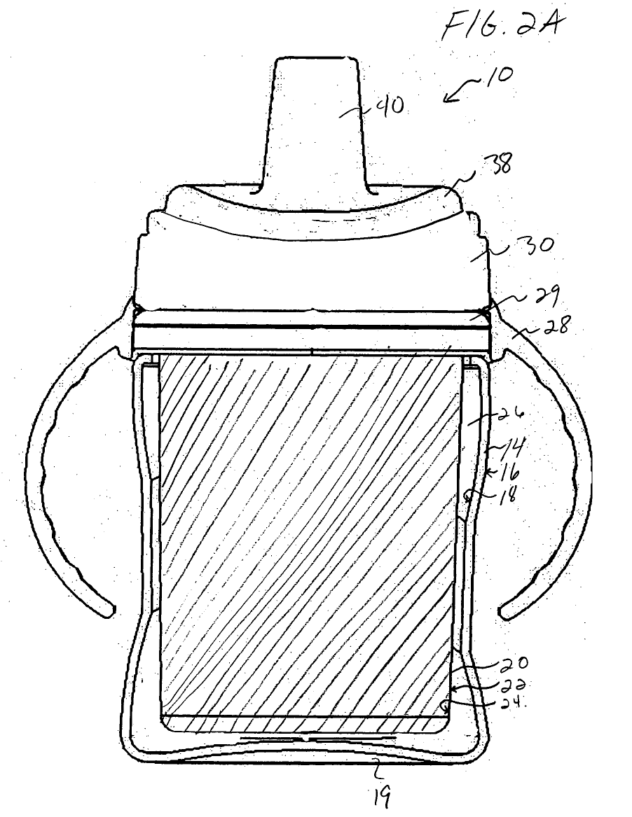

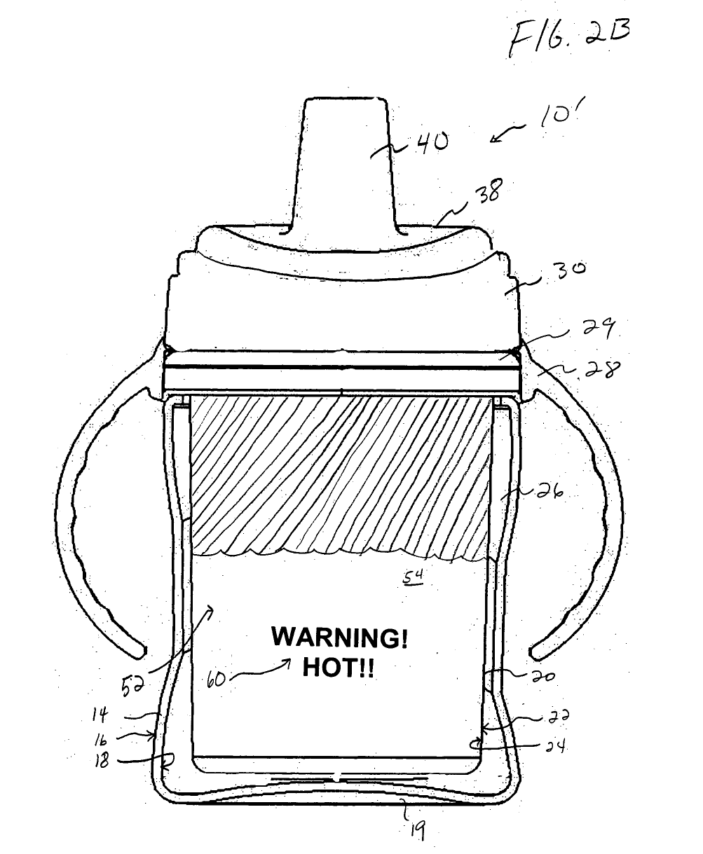

FIGS. 2A and 2B illustrate an alternate implementation of a drinking cup 10′. Similar to the drinking cup 10 described above with respect to FIG. 1, the drinking cup 10′ includes an outer cup 12 having a bottom surface 19. The outer cup 12 includes an outer wall 14 and a bottom surface 19. The outer wall 14 includes an exterior surface 16 and an interior surface 18. An inner wall 20 is disposed within the interior surface 18 and includes an outer surface 22 and an inner surface 24.

The drinking cup 10′ also includes a thermo-chromatic ink 52 or other suitable thermo-chromatic substance. Upon the introduction of a beverage 54 into the inner surface 24 of the inner wall 20, the thermo-chromatic ink displays a color variance. A message 60 may be displayed upon the introduction of a beverage that has a temperature greater than the temperature threshold of the thermo-chromatic ink 52. For example, if the drinking cup 10′ is being used for baby formula, which must be delivered to the child at a temperature warm enough to satisfy the baby but not too warm to burn or irritate the child, the thermo-chromatic substance may only show the message 60 when the beverage has a higher temperature than the temperature threshold of the thermo-chromatic ink 52. For example, a baby formula may be too hot for a child at a temperature greater than 110° F. The thermo-chromatic ink 52 may be impregnated within the inner wall 20, or may be affixed to the outer surface 22 of the inner wall 20. Alternatively, the message 60 may be formed by molding the inner wall 20 with an inkless portion or portions that do not exhibit a color variance, so that the arrangement of the inkless portion forms the message 60. Thus, when the thermo-chromatic substance exhibits a color variance, the portions of the inner wall 20 that are not impregnated with the thermo-chromatic ink 52 display the message 60.

A number of embodiments of the invention have been described. Nevertheless, it will be understood that various modifications may be made without departing from the spirit and scope of the invention. For example, the thermo-chromatic ink may be adapted to show multiple color variances corresponding to multiple temperature thresholds. Alternatively, multiple variations of thermo-chromatic ink may be used so that multiple thermo-chromatic temperature thresholds exist in the drinking cup. Accordingly, other embodiments are within the scope of the following claims.

1. A drinking cup comprising:
an inner wall having an outer surface and an inner surface, wherein the inner wall includes a thermo-chromatic substance, the thermo-chromatic substance operable to exhibit a color variance corresponding to a temperature variance in a beverage in the cup and in contact with the inner wall;
an outer wall disposed about the inner wall, the outer wall having a transparent portion adapted to permit a visual perception of at least a portion of the inner wall through the outer wall, wherein the visual perception includes exhibition of the color variance; and
a thermal insulation layer disposed between the inner wall and the outer wall.

2. The drinking cup of claim 1, wherein the thermal insulation layer comprises air.

3. The drinking cup of claim 2, wherein the thermal insulation layer comprises a space between the inner wall and at least a portion of the outer wall, wherein the pressure of the air in the thermal insulation layer is lower than the pressure external to the thermal insulation layer.

4. The drinking cup of claim 1, wherein the thermo-chromatic substance is contained within the inner wall

5. The drinking cup of claim 1, wherein the thermo-chromatic substance is disposed in a layer disposed on the outer surface of the inner wall.

6. The drinking cup of claim 1, further comprising a lid, wherein the lid includes a spout, the lid adapted to prevent a beverage disposed within the inner wall from exiting a space bounded by the inner wall and the lid except through the spout.

7. The drinking cup of claim 6, further comprising an air vent disposed in the lid, the air vent operable to permit the flow of the liquid out of the space bounded by the inner wall and the lid through the spout.

8. The drinking cup of claim 1, wherein the transparent portion permits a visual perception of the outer surface of the entire inner wall.

9. The drinking cup of claim 1, wherein the outer wall comprises a wall of an outer cup that also forms a bottom surface of the drinking cup.

10. The drinking cup of claim 9, wherein the outer cup is a unitary piece of resin with molded surfaces.

11. The drinking cup of claim 10, wherein the outer cup is molded of transparent resin.

12. The drinking cup of claim 9, wherein the inner wall comprises a wall of an inner cup spaced apart from the outer cup over substantially an entire extent of the inner cup save for an upper end of the cup.

13. The drinking cup of claim 12, wherein the inner cup is a unitary piece of resin with molded surfaces, the resin of the inner cup containing the thermo-chromatic substance.

14. The drinking cup of claim 1, wherein the drinking cup is sized for grasping by a child of about six months old.

15. The drinking cup of claim 1, wherein the drinking cup defines an interior volume of between about 5 ounces and about 10 ounces.

16. The drinking cup of claim 1, further comprising a graphic design disposed on a layer affixed to the outer surface of the inner wall.

17. The drinking cup of claim 1, further comprising a graphic design disposed within the inner wall, wherein the graphic design is formed by an absence of the thermo-chromatic material, the graphic design visible only when the inner wall displays thermo-chromatic properties.

18. The drinking cup of claim 1, wherein the thermo-chromatic material displays the color variance when the beverage placed in contact with the inner wall has a temperature lower than the temperature of the inner wall prior to placing the beverage in contact with the inner wall.

19. The drinking cup of claim 1, wherein the thermo-chromatic material displays the color variance when the beverage placed in contact with the inner wall has a temperature higher than the temperature of the inner wall prior to placing the beverage in contact with the inner wall.

20. The drinking cup of claim 17, wherein the thermo-chromatic material displays a color variance when the beverage placed in contact with the inner wall has a temperature higher than the temperature of the inner wall prior to placing the beverage in contact with the inner wall.

21. The drinking cup of claim 20, wherein the design layer disposed in the inner wall communicates a warning message that the beverage temperature is higher than recommended for consumption by a child.

22. The drinking cup of claim 21, wherein the thermo-chromatic substance exhibits a color variance when the beverage temperature is greater than about 110° F.

23. The drinking cup of claim 1, wherein the thermo-chromatic substance includes multiple temperature thresholds, each of the temperature thresholds operable to impart a different color variance from the other temperature thresholds.
